(19) United States
(12) Patent Application Publication
(10) Pub. No.: US 2024/0285010 A1
(43) Pub. Date: Aug. 29, 2024

(54) COATING AND FABRIC USING ANTIMICROBIAL POROUS MEMBRANE

(71) Applicants: ENERGYFRONT INC., Okayama-shi (JP); Shinagawa General Co., Ltd., Tokyo (JP); SCHOOL CORPORATION SHUJITSUGAKUEN, Okayama-shi (JP)

(72) Inventors: Takeji UEDA, Okayama-shi (JP); Hajime SUZUKI, Moka-shi (JP); Masayasu SEKI, Moka-shi (JP); Sumiko SIOTA, Okayama-shi (JP); Yoichi YAMADA, Okayama-shi (JP)

(21) Appl. No.: 18/073,930

(22) Filed: Dec. 2, 2022

Related U.S. Application Data
(63) Continuation of application No. PCT/JP2021/016405, filed on Apr. 23, 2021.

(30) Foreign Application Priority Data
Jun. 5, 2020 (JP) 2020-098915

(51) Int. Cl. A41D 31/30; A61K 31/192; B01D 39/16

(52) U.S. Cl. CPC A41D 31/30; A61K 31/192; B01D 39/16

(57) ABSTRACT

According to an embodiment of the present invention, a coating comprising an antimicrobial porous membrane, the porous membrane including an allophane particle and an abietane-type diterpenoid compound adsorbed on the allophane particle is provided. In addition, according to another embodiment of the present invention, a fabric comprising a base material which is a fabric, and an antimicrobial porous membrane arranged on the base material is provided. The antimicrobial porous membrane includes an allophane particle and an abietane-type diterpenoid compound adsorbed on the allophane particle.

### CROSS-REFERENCE TO RELATED APPLICATIONS

This application is a Continuation of International Patent Application No. PCT/JP2021/016405, filed on Apr. 23, 2021, which claims the benefit of priority to Japanese Patent Application No. 2020-098915, filed on Jun. 5, 2020, the entire contents of which are incorporated herein by reference.

### FIELD

The present invention relates to a coating and a fabric using an antimicrobial porous membrane that can be applied to a tool, clothes, a building material, or the like used in a medical or nursing site or in daily life.

### BACKGROUND

Sterilization and antimicrobial are important problems in medical and nursing sites. For this reason, heat sterilization, disinfection with alcohol disinfectant, iodine disinfectant, hypochlorous acid disinfectant, phenolic disinfectant, and surfactant disinfectant, silver ion antimicrobial treatment, ultraviolet irradiation, photocatalyst processing, and the like are applied to equipment used in medical and nursing sites. These methods are used depending on the environment in which they are used and the target substances.

A method of temporarily imparting sterilization properties and antimicrobial properties to the surface of the human body, furniture, and the like, such as spraying alcohol or wiping with a fabric containing a disinfectant, may be adopted. Surgical tools, sheets, and the like are sterilized by a dedicated autoclave or sterilization gas treatment equipment.

As with sheets, clothes can be sterilized in an autoclave or sterilization gas treatment equipment. However, in general households and nursing facilities, sterilization by washing with bleach is the mainstream. In addition, there are materials having antimicrobial properties among natural materials such as fibers made of bamboo, and such natural materials are sometimes utilized for antimicrobial equipment.

For the object unsuitable for wiping, washing, etc., the material itself is devised to have antimicrobial properties. For example, a method of binding an antimicrobial substance to a fabric with a binder disclosed in Japanese laid-open patent publication No. 2014-055394 and Japanese laid-open patent publication No. 2014/102980 is used for items that are infrequency washed and have complex shapes, such as medical equipment or heat-resistant clothes for firefighting. Similarly, for a handrail, door knob, or the like, a plastic obtained by kneading silver is used as a material, or a film to which an antimicrobial is added is laminated on a base material.

As described above, disinfectants and antimicrobials are frequently used in various ways in hospitals and the like. However, drug-resistant bacteria (e.g., methicillin-resistant Staphylococcus aureus (MRSA)) develop in this environment and may be problematic as a cause of nosocomial infections in hospitals. In recent years, community-acquired MRSA has also become widespread, and countermeasures have been required. Conventional disinfectants and antimicrobials do not function effectively against bacteria that have acquired chemical resistance, such as MRSA.

The efficacy of various naturally occurring substances has been shown against drug-resistant Staphylococcus aureus and the like. For example, there are abietane-type diterpenoid compounds shown in International patent publication No. WO2010/119638 and International patent publication No. WO2016/051013. They are effective in inhibiting the formation of biofilms (structure bodies in which bacterial products such as microorganisms and extracellular polysaccharides gather on the surface of a solid, which gain favorable living conditions for bacteria and increase the number of them) and are effective in antimicrobial activities regardless of the presence or absence of drug resistance.

### SUMMARY

According to an embodiment of the present invention, a coating comprising an antimicrobial porous membrane, the porous membrane including an allophane particle and an abietane-type diterpenoid compound adsorbed on the allophane particle is provided.

The abietane-type diterpenoid compound may include at least one selected from among abietic acid, dehydroabietic acid and neoabietic acid.

The antimicrobial porous membrane may further include an inorganic salt and/or at least one metal ion adsorbed on the allophane particle.

The inorganic salt and/or the at least one metal ion may have an antimicrobial property.

The at least one metal ion may include at least one of platinum ion, silver ion and copper ion.

The abietane-type diterpenoid compound may be adsorbed directly onto the allophane particle without a holding material.

According to an embodiment of the present invention, a fabric comprising a base material, the base material being a fabric, and the antimicrobial porous membrane, which is described above, provided on the base material.

The base material may be a nonwoven fabric.

### BRIEF DESCRIPTION OF DRAWINGS

FIG. 1 is a diagram showing a porous membrane according to an embodiment of the present invention.

FIG. 2 is a diagram showing a structure of a unit particle of allophane.

FIG. 3 is a diagram showing a porous membrane containing allophane, which is arranged on a base material using an AD method.

FIG. 4 is a diagram showing a porous membrane using an adhesion layer.

FIG. 5 is a diagram showing biofilm formation inhibition amounts for allophane membranes and nonwoven fabrics measured by a CV method and their approximate curves of exponential functions.

FIG. 6 is a diagram showing biofilm formation inhibition amounts for allophane membranes and nonwoven fabrics measured by a WST method and their approximate curves of exponential functions.

FIG. 7 is a diagram showing a biofilm formation inhibitory activity of an allophane membrane and a nonwoven fabric and their approximate curves of exponential functions.

FIG. 8 is a diagram showing a biofilm formation inhibitory activity of an allophane membrane and a nonwoven fabric and their approximate curves of exponential functions.

FIG. 9 is a diagram showing a biofilm formation inhibitory activity of an allophane membrane and a nonwoven fabric and their approximate curves of exponential functions.

### DESCRIPTION OF EMBODIMENTS

As described above, various sterilization means or antimicrobial means are known, and various drugs have been studied in order to cope with various bacteria. However, there are limited ways to easily and stably hold inorganic substances or organic molecules having these bactericidal or antimicrobial properties on a desired solid surface, in particular a flexible material such as fabric or a three-dimensional structure such as a door knob. Since the above-described biofilm is formed in a place where moisture is high, there is a demand for a method of stably attaching antimicrobial molecules that inhibit the formation of biofilm to surfaces of various objects such as an instrument around water, a door knob touched by a hand of a person, clothes and footwear that come in contact with a sweat-inducing portion, and the like.

It is generally difficult to stably immobilize various organic molecules having bactericidal or antimicrobial properties. In the method disclosed in Japanese laid-open patent publication No. 2014-055394, adding polyhexamethylene biguanide hydrochloride and the like to yarns and fabrics by using a binder resin in the dyeing and/or finishing step is exemplified. However, the method using a binder tends to cause the organic molecules to be buried in the binder and lose the effect. It is technically possible to apply a silane coupling treatment to a fabric to attach organic molecules without using a binder or to synthesize a polymer having a molecular structure having antimicrobial properties and coat the polymer on the fabric. However, it is necessary to find an appropriate attachment method depending on the molecular structure of the organic molecule and the type of base material to which the organic molecule is attached, and mass production is costly.

Similarly, in the method disclosed in Japanese laid-open patent publication No. 2014/102980, an inorganic antimicrobial material such as a silver-supported inorganic porous material or a zinc-supported inorganic porous material is attached to a fabric with a binder resin. In this case as well, although thickening the binder increases the immobilization of an antimicrobial material, the antimicrobial material is more likely to be embedded in the binder. Since the antimicrobial properties depend on how much the antimicrobial material is exposed to the surface, once the antimicrobial material is buried in the binder, the antimicrobial properties of the antimicrobial decrease.

According to the present invention, it is possible to provide a versatile porous membrane capable of easily fixing an inorganic substance or an organic molecule having various bactericidal properties, antimicrobial properties, and the like to a desired solid surface, and a fabric or a three-dimensional structure provided with the porous membrane.

In an embodiment of the present invention, various inorganic substances or organic molecules having bactericidal or antimicrobial properties are immobilized on a porous material with high adsorption capacity. Coating the porous material on a base material forms an antimicrobial porous membrane, and a coating with the antimicrobial porous membrane is formed on the base material.

Various materials can be used as the particulate porous material. The porous material generally adsorbs both hydrophilic molecules and hydrophobic molecules, and therefore has high versatility as a support means. Examples of a material having a spherical microstructure, good adhesion as a film, and capable of holding various molecules include an allophane having a relatively large hole diameter. The allophane can hold a variety of ions and atoms in addition to molecules. The allophane will be described later. Also, the porous material used in an embodiment of the present invention is not limited to allophane, and various porous materials such as zeolite, titania, carbon, silica, and glass can be used.

The inorganic substance or the organic molecule can be supported within the fine hole of the porous material by coating a solution containing an inorganic substance or an organic molecule having bactericidal or antimicrobial properties on the porous material to attach the inorganic substance or the organic molecule and then evaporating the solvents. Some organic molecules exhibiting bactericidal or antimicrobial properties have a large hydrophobic group and a hydrophilic group at the ends. Therefore, in the case where a solution containing an organic molecule is prepared, water or an organic solvent capable of dissolving the organic molecule is used as the solvent.

Inorganic or organic molecules-supporting porous materials having bactericidal or antimicrobial properties can be coated on a base material surface by contacting the base material without the use of a binder. The particulate porous material has momentum.

A versatile antimicrobial porous membrane is produced by the above method. Applying the antimicrobial porous membrane to the fabric makes it possible to make an antimicrobial fabric or coating the antimicrobial porous membrane on a three-dimensional structure such as a door knob makes it possible to make an antimicrobial door knob.

Hereinafter, a porous membrane according to an embodiment of the present invention will be described in detail with reference to the drawings.

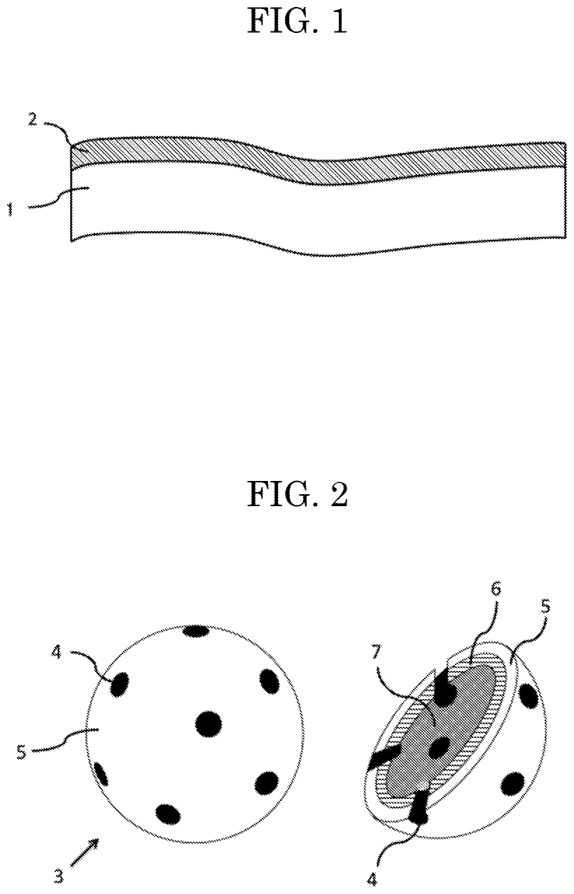

FIG. 1 shows a porous membrane according to an embodiment of the present invention. As shown in FIG. 1, a porous membrane 2 is formed by depositing porous particles on a surface of a base material 1. In the present embodiment, the base material 1 may be a hard solid such as metal, wood, and plastic, as well as a deformable solid such as fabric, rubber sheet, sponge, and aluminum foil.

Various known materials can be used as the porous particle constituting the porous membrane 2. For example, activated carbon, zeolite, titania, allophane, mesoporous silica, mesoporous alumina, porous glass, porous metal, metal complex porous material, and the like can be used as the porous particle. In the present embodiment, the case where allophane is used as the porous particle will be described as an example.

Allophane is a low-crystallinity aluminum silicate and an amorphous aluminum silicate that are abundantly found in soils derived from volcanic products such as pumice and volcanic ash. Allophane consists of silicon (Si), aluminum (Al), oxygen (O), and hydrogen (H) (hydroxyl groups (OH)). FIG. 2 is a diagram showing a structure of a unit particle of allophane 3. The unit particle of allophane 3 has a hollow 7 inside, an octahedral sheet 5 similar to Al(OH)₃ gibbsite as an outer shell, and a SiO₄ tetrahedral sheet 6 as the inner shell. The unit particle of allophane 3 is a hollow-spherical particle with a diameter of 3.5 nm to 5 nm and more particularly has a specific surface area (≤900 m²/g). The unit particle of allophane 3 has a structure in which the gibbsite octahedral sheet of a single layer is formed as a spherical wall, and SiO₄ tetrahedron is bonded to the inside thereof, and many fine holes 4 having a hole diameter of 0.3 nm to 0.5 nm are present in the spherical wall. Because of such a structure, allophane has a large surface area and a hydroxyl group on the surface, so that water, ions, organic substances, various gas components, and the like can be adsorbed. Although allophane is a natural clay mineraloid, it can also be artificially made.

The porous membrane 2 functions as an antimicrobial film by supporting ions, atoms, or molecules having bactericidal or antimicrobial properties on the porous membrane 2. The bactericidal substance or the antimicrobial substance may be fixed to the porous particles before the film formation of the porous membrane 2, or may be fixed after the film formation. Allophane can directly adsorb ions, atoms, or molecules having bactericidal or antimicrobial properties without any holding material such as a capsule or a gel. Therefore, allophane can constitute the porous membrane 2 in which functional molecules are directly adsorbed. However, depending on the type of the adsorbed molecules, the molecule adsorbs not only on the fine hole but also on a particle surface and it may be difficult to form a film by depositing without a binder, which will be described later.

In the case where a water-soluble salt containing at least one metal ion having antimicrobial properties, such as silver ion, copper ion, or platinum ion, or a highly hydrophilic surfactant is supported on the porous membrane 2, the porous particles before film formation or the porous membrane 2 with porous particles deposited are immersed in aqueous solution. In the case where a hydrophobic substance is supported on the porous membrane 2, a solution containing the hydrophobic substance is prepared using an organic solvent in which a hydrophobic substance such as acetone, ethanol, or ethyl ether is soluble, and the porous particles before film formation or the porous membrane 2 are immersed in the solution. In the case where a substance having a low boiling point and capable of being gasified is supported on the porous membrane 2, a gas containing the vaporized substance is adsorbed onto the porous particles before film formation or the deposited porous membrane 2.

Various methods are known as a method of forming the porous membrane 2 on the base material 1 without using a binder. For example, a porous polymer membrane can be made utilizing phase separation. The porous metal may be produced by blowing a gas into molten metal. In addition, porous silica can be formed by a sol-gel method or the like.

However, these methods often require special conditions, which may alter and destroy the material. Therefore, it is desired to form a porous membrane by depositing porous particles without losing their properties and without damaging the material.

An aerosol deposition method (AD method) is known as a method of depositing fine particles. Aerosol is a mixture of air or inert gas and fine particles. The AD method is a method in which the aerosol is sprayed from a nozzle toward a base material to collide with the base material, and a film containing the fine particles is directly formed on the base material. In the case where the porous membrane 2 is formed by the AD method, it can be performed at room temperature, and film formation can be performed without damaging the material, without impairing the properties of the porous particles as raw material, and without using binders.

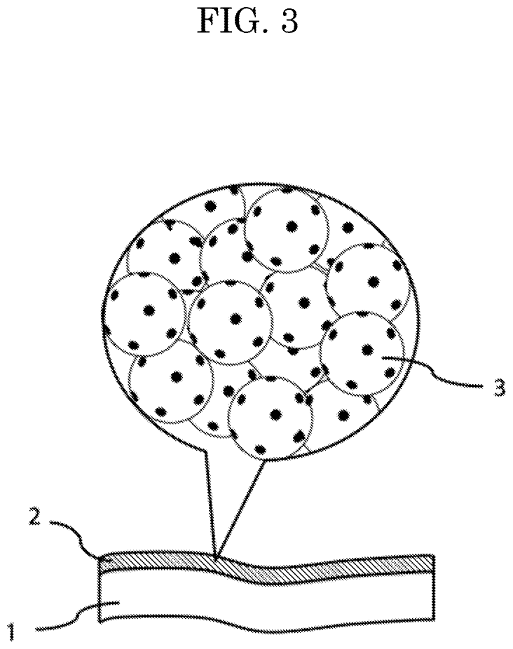

According to studies conducted by the present inventors, although the particle structure of allophane is destroyed by means such as sputtering, allophane can be deposited on the base material 1 without destroying its particle structure when using the AD method. FIG. 3 is a diagram showing the porous membrane 2 containing allophane, which is arranged on the base material 1 using the AD method.

There are a variety of substances that are supported on the porous membrane 2 and have bactericidal or antimicrobial properties. For example, effects of an abietane-type diterpenoid compound such as abietic acid, a monocyclic monoterpene such as hinokitiol, a sucrose fatty acid ester, and the like have been confirmed as a biofilm formation inhibitor effective as an MRSA in addition to silver ions. Many of these organic compounds having the ability to inhibit biofilm formation have a structure with hydrophilic group at the end of a large hydrophobic group serving as a skeleton and are easily supported by the porous material described above.

In particular, the abietane-type diterpenoid compounds are suitable as biofilm formation inhibitors for MRSA. In addition to abietic acid shown below, neoabietic acid, dehydroabietic acid, and the like are expected to be effective as biofilm formation inhibitors as the abietane-type diterpenoid compound.

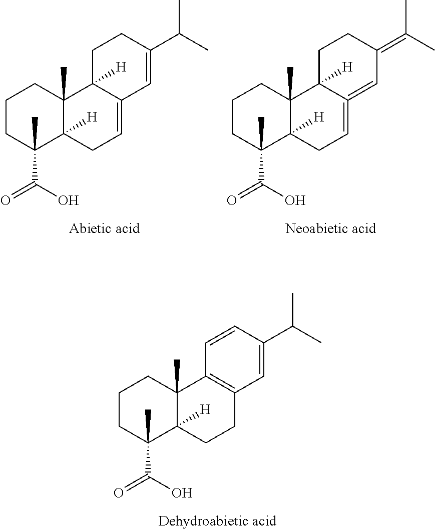

Also, in the case where the porous membrane 2 is produced, not only one type of porous particle but also a plurality of different types of porous particles may be used to form a deposition film. In addition, not only one substance having bactericidal or antimicrobial properties is supported on the porous particles, but also a plurality of substances having different bactericidal or antimicrobial properties may be supported on the porous particles. For example, in addition to the biofilm formation inhibitors effective as the MRSA described above, yomogi extract, metal ions having antimicrobial properties, such as silver ions, copper ions, and platinum ions, inorganic salts having antimicrobial properties, containing these metal ions, biofilm formation inhibitors of other pathogenic bacteria, molecular compounds effective as antiviral agents against pathogenic viruses, and the like may be supported on the porous particles.

In the case where the porous membrane 2 is formed by the AD method, the adhesion may be inferior depending on the combination of the chemical properties of the base material and the particles. In this case, it is effective as a method of increasing adhesion without using a binder to form aerosol from different types of particles and use it, or to arrange an adhesion layer 9 on the base material 1 before forming the porous membrane 2.

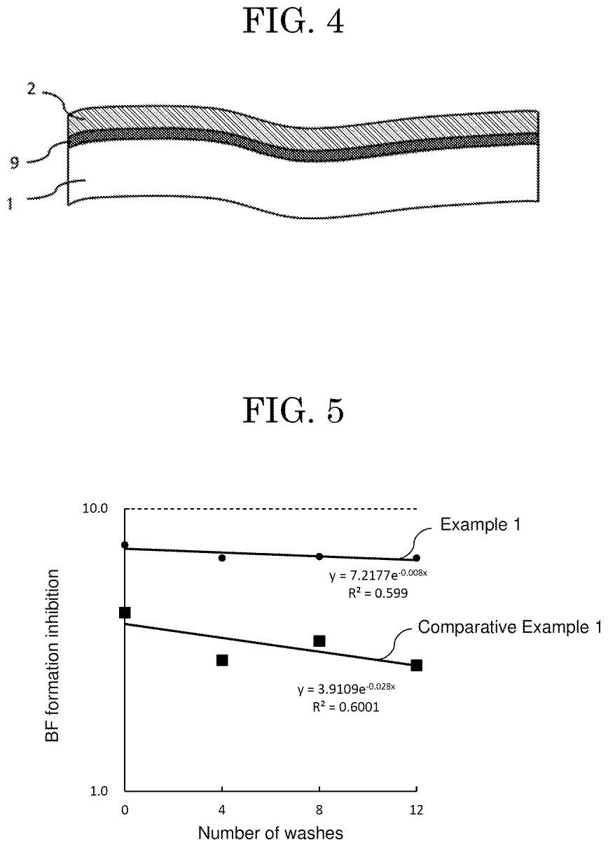

FIG. 4 shows the case where the adhesion layer 9 is disposed between the porous membrane 2 and the base material 1 according to an embodiment of the present invention. The material of the adhesion layer 9 is selected according to the material of the base material 1 to be used and the chemical properties of the porous particles contained in the porous membrane 2, and for example, polyethylene, polypropylene, polyvinyl alcohol, polyamide resin, or the like may be used. Although the adhesion layer 9 can be formed on the base material 1 by an ink jet method, a coating method, the AD method, or the like, the method of forming the adhesion layer 9 is not limited to these methods. Arranging the porous membrane 2 on the base material 1 via the adhesion layer 9 improves the adhesion strength between the base material 1 and the porous membrane 2 due to the anchor effect of the adhesion layer 9. In this case, unlike the binder conventionally used, the adhesion layer 9 does not cover the substance supported by the porous particles constituting the porous membrane 2. Therefore, the adhesion strength between the base material 1 and the porous membrane 2 can be improved without impairing the function of the substance having bactericidal or antimicrobial properties.

The bactericidal or antimicrobial porous membrane 2 according to an embodiment of the present invention is applicable to various fields. For example, the porous membrane 2 can be applied to a place where biofilm is likely to be formed, such as around water, a door knob or an electric switch, a keyboard, or a handrail touched by a hand of a person, clothes or side pads that come in contact with sweat, a seat surface such as a toilet seat or the automobile, a filter such as a water purifier or an air conditioner, and the like.

In addition, in the case where the base material is a fabric, if an antimicrobial fabric is made by forming a bactericidal or antimicrobial porous membrane on the fabric, the antimicrobial fabric can be applied to various things such as clothes, building materials, and packaging materials.

### EXAMPLES

Hereinafter, although the present invention will be described in detail based on Examples, the present invention is not limited to these Examples.

### Antimicrobial Activity Persistence Test of Dehydroabietic Acid-Containing Allophane Membrane

### Example 1

Allophane raw material fine particles are sprayed onto a nonwoven fabric base material coated with a polyethylene film to form an allophane membrane by the AD method. First, sieved allophane powder was aerosolized with nitrogen gas and dry air at a flow rate of 2.4 L/min. Then, the aerosolized allophane was sprayed through a nozzle with opening wides of 7.0 mm×0.4 mm, 30 mm×0.2 mm, 10 mm×0.1 mm onto a nonwoven base material placed in a chamber of 60 Pa to 120 Pa vacuum atmosphere to form an allophane membrane, thereby preparing an allophane membrane-nonwoven fabric composite. The nozzle was reciprocated while being displaced at a speed of 40 mm/s to 2.5 mm/s with respect to the base material, the film formation time was set to 4 minutes to 7 minutes, and the film formation area was set to 75×75 mm².

A dehydroabietic acid solution (solvent: acetone) was uniformly added dropwise to the deposited allophane membrane (1 cm×1 cm) and dried overnight (final concentration: 25 μg/cm²). A plurality of dehydroabietic acid-supported allophane membranes was prepared by the above method.

The produced dehydroabietic acid-supported allophane membranes were washed. The number of washes was 0, 4, 8, and 12, respectively. The washing was carried out by immersing each of the dehydroabietic acid-supported allophane membranes in 0.5 mL purified water for 30 minutes together with the base material, pulling up the dehydroabietic acid-supported allophane membrane after each washing and immersing it in fresh purified water in the same manner.

Next, an S. aureus N315 strain was added to a brain heart infusion (BHI) medium to which 1% glucose was added, the dehydroabietic acid-supported allophane membranes washed 0 times, 4 times, 8 times, and 12 times were immersed in the BHI medium together with the base materials, and each of the base materials was pressed with a stainless-steel circular washer and allowed to stand at 37° C. for 24 hours.

Thereafter, the biofilm formation amount of each of the allophane membranes was measured. Specifically, the amounts of biofilm formed on the surfaces of the allophane membranes differing in the number of washes were measured by a CV method and a WST method, and the biofilm formation amount of each allophane membrane was calculated. The CV method and the WST method will be described later.

### Comparative Example 1

In a comparative example, a dehydroabietic acid solution (solvent: acetone) was uniformly added dropwise to a nonwoven fabric (1 cm×1 cm) and dried overnight (final concentration: 25 μg/cm²). A plurality of dehydroabietic acid-supported nonwoven fabrics was prepared and washed 0 times, 4 times, 8 times, and 12 times, respectively, in the same manner as the allophane membrane. After that, an S. aureus N315 strain was added to the BHI medium to which 1% glucose was added, and each of the dehydroabietic acid-supported nonwoven fabrics was immersed in the BHI medium, pressed with a stainless-steel circular washer, and allowed to stand at 37° C. for 24 hours. Thereafter, the biofilm formation amounts of the nonwoven fabrics were measured by the CV method and the WST method in the same manner as the allophane membranes of Example 1.

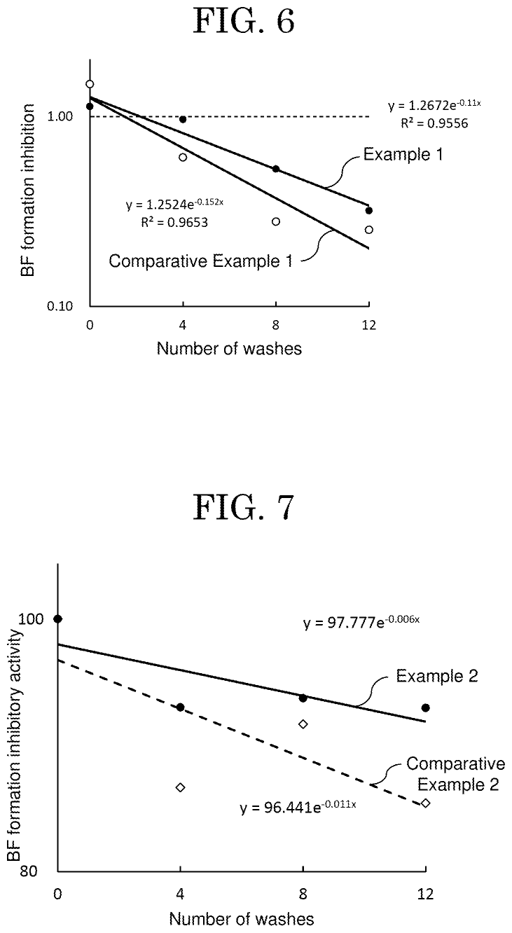

FIG. 5 and FIG. 6 show the biofilm formation inhibition amounts of the dehydroabietic acid-supported allophane membranes (Example 1) and the biofilm formation inhibition amounts of a dehydroabietic acid-supported nonwoven fabrics (Comparative Example 1) without forming an allophane membrane. FIG. 5 shows biofilm formation inhibition amounts and their approximate curves of exponential functions of allophane membranes and nonwoven fabrics measured by the CV method. FIG. 6 shows biofilm formation inhibition amounts and their approximate curves of exponential functions of allophane membranes and nonwoven fabrics measured by the WST method. Hereinafter, the procedure of the CV method and the WST method will be described in detail.

The CV method will be described. First, the step of immersing the dehydroabietic acid-supported allophane membrane of Example 1 and the dehydroabietic acid-supported nonwoven fabric of Comparative Example 1 in a container containing a large amount of purified water and washing them was repeated twice. Next, the dehydroabietic acid-supported allophane membrane of Example 1 and the dehydroabietic acid-supported nonwoven fabric of Comparative Example 1 were immersed in 0.5 ml of 0.1 mass % crystal violet (CV) aqueous solution for 15 minutes. Thereafter, the step of immersing the dehydroabietic acid-supported allophane membrane of Example 1 and the dehydroabietic acid-supported nonwoven fabric of Comparative Example 1 in a container containing a large amount of purified water and washing them was repeated five times. Thereafter, the dehydroabietic acid-supported allophane membrane of Example 1 and the dehydroabietic acid-supported nonwoven fabric of Comparative Example 1 were dried for several hours. The dried dehydroabietic acid-supported allophane membrane of Example 1 and the dehydroabietic acid-supported nonwoven fabric of Comparative Example 1 were immersed in a 30 vol % acetic acid solution, respectively, to elute CV that dyed the biofilm formed on the dehydroabietic acid-supported allophane membrane of Example 1 and the dehydroabietic acid-supported nonwoven fabric of Comparative Example 1. The amount of CV eluted in the acetic acid solution was quantified by measuring the absorbance at 570 nm of the acetic acid solution.

The WST method will be described. First, the step of immersing the dehydroabietic acid-supported allophane membrane of Example 1 and the dehydroabietic acid-supported nonwoven fabric of Comparative Example 1 in a container containing a large amount of purified water and washing them was repeated seven times. Next, the dehydroabietic acid-supported allophane membrane of Example 1 and the dehydroabietic acid-supported nonwoven fabric of Comparative Example 1 were immersed in a mixed solution of 25 μL of a WST mixed solution (M439, manufactured by DOJINDO LABORATORIES) and 475 μL of the BHI medium, and allowed to stand at 37° C. for 1 hour. Thereafter, 50 μL of a mixed solution was collected from 500 μL of WST mixed solution and BHI medium, and the absorbance at 450 nm of the collected mixed solution was measured to quantify the biofilm formation amounts of the dehydroabietic acid-supported allophane membrane of Example 1 and the dehydroabietic acid-supported nonwoven fabric of Comparative Example 1.

Referring to FIG. 5, the slope of the approximate curve was −0.008 for the allophane membrane and −0.028 for the nonwoven fabric. In addition, referring to FIG. 6, the slope of the approximate curve was −0.11 for the allophane membrane and −0.152 for the nonwoven fabric. Therefore, it was shown that dehydroabietic acid-supported allophane membranes were more effective in inhibiting biofilm formation than nonwoven fabric even after repeated washings in measurements by both the CV and WST methods. Therefore, it was shown that the allophane membrane has a high capacity to hold dehydroabietic acid.

### <Dehydroabietic Acid Elution Experiment>

The elution experiment of dehydroabietic acid was carried out for the solvent of the dehydroabietic acid-supported allophane membrane.

### [Preparation of Allophane Membrane by AD Method]

First, a dehydroabietic acid-supported allophane membranes were prepared. Each of the allophane membranes was produced by the AD method by spraying allophane raw material fine particles onto a nonwoven fabric base material coated with a polyethylene film as in Example 1 described above. First, sieved allophane powder was aerosolized with nitrogen gas and dry air at a flow rate of 1.2 to 4.8 L/min and the aerosolized allophane was sprayed through a nozzle with opening wides of 7.0 mm×0.4 mm, 30 mm×0.2 mm, 10 mm×0.1 mm, 10 mm×0.6 mm onto a nonwoven base material placed in a chamber of 60 Pa to 120 Pa vacuum atmosphere to form an allophane membrane, thereby preparing an allophane membrane-nonwoven fabric composite. The nozzle was reciprocated while being displaced at a speed of 40 mm/s to 2.5 mm/s with respect to the base material, the film formation time was set to 4 minutes to 7 minutes, and the film formation area was set to 75×75 mm².

A dehydroabietic acid solution (solvent: acetone) was uniformly added dropwise to the deposited allophane membrane (1 cm×1 cm) and dried overnight (final concentration: 25 μg/cm²). A plurality of dehydroabietic acid-supported allophane membranes was prepared by the above method.

### Example 2

The dehydroabietic acid-supported allophane membranes prepared by the above-described AD method were washed with purified water. The number of washes was 0, 4, 8, and 12, respectively. The washing was carried out by immersing each dehydroabietic acid-supported allophane membrane in 0.75 ml purified water together with the base material and allowing it to stand for 30 minutes. Washing was carried out by pulling up each of the dehydroabietic acid-supported allophane membranes after each washing and immersing it in fresh purified water in the same manner.

Next, the brain heart infusion (BHI) medium to which 1% glucose was added was prepared, an S. aureus N315 strain was added to the BHI medium, the dehydroabietic acid-supported allophane membranes washed 0 times, 4 times, 8 times, and 12 times were immersed in the BHI medium together with the base materials, and each of the base materials was pressed with a stainless-steel circular washer and allowed to stand at 37° C. for 24 hours.

The biofilm formation amount of each of the allophane membranes was measured. Specifically, the amounts of biofilm formed on the surfaces of the allophane membranes differing in the number of washes were measured by the CV method, and the biofilm formation inhibitory activity of each allophane membrane was calculated. The result is shown in FIG. 7.

### Comparative Example 2

A dehydroabietic acid solution (solvent: acetone) was uniformly added dropwise to the nonwoven fabric (1 cm×1 cm) and dried overnight (final concentration: 25 μg/cm²). A plurality of dehydroabietic acid-supported nonwoven fabrics was prepared, and washed 0 times, 4 times, 8 times, and 12 times with purified water respectively in the same manner as in Example 2. After that, an S. aureus N315 strain was added to the BHI medium to which 1% glucose was added, and each of the dehydroabietic acid-supported nonwoven fabrics was immersed in the BHI medium, pressed with a stainless-steel circular washer, and allowed to stand at 37° C. for 24 hours. Thereafter, the biofilm formation inhibitory activity of the nonwoven fabrics was measured in the same manner as the allophane membrane of Example 2. The result is shown in FIG. 7.

### Example 3

The dehydroabietic acid-supported allophane membranes prepared by the AD method described above were washed with 70% ethanol (v/v). The number of washes was 0, 2, 4, 6, and 8, respectively. The washing was carried out by immersing each dehydroabietic acid-supported allophane membrane in 0.75 mL ethanol together with the base material and allowing it to stand for 30 minutes. Washing was carried out by pulling up each of the dehydroabietic acid-supported allophanes membrane after each washing and immersing it in fresh ethanol in the same manner.

Next, an S. aureus N315 strain was added to the BHI medium to which 1% glucose was added, the dehydroabietic acid-supported allophane membranes washed 0 times, 2 times, 4 times, 6 times, and 8 times were immersed in the BHI medium together with the base materials, and each of the base material was pressed with a stainless-steel circular washer and allowed to stand at 37° C. for 24 hours.

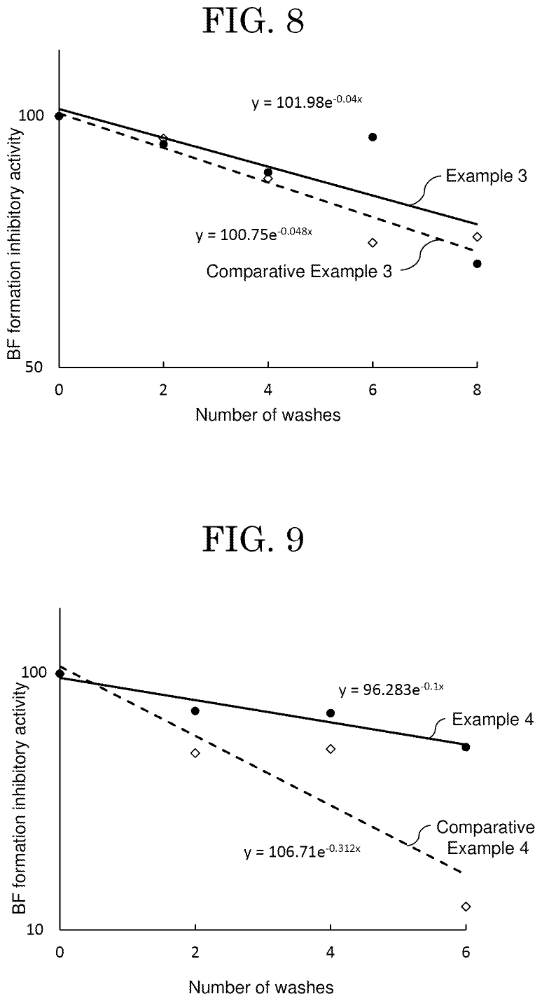

The biofilm formation amount of each of the allophane membranes was measured. Specifically, the amounts of biofilm formed on the surfaces of the allophane membranes differing in the number of washes were measured by the CV method, and the biofilm formation inhibitory activity of each allophane membrane was calculated. The result is shown in FIG. 8.

### Comparative Example 3

A dehydroabietic acid solution (solvent: acetone) was uniformly added dropwise to the nonwoven fabric (1 cm×1 cm) and dried overnight (final concentration: 25 μg/cm²). A plurality of dehydroabietic acid-supported nonwoven fabrics was prepared and washed 0 times, 2 times, 4 times, 6 times, and 8 times with 70% ethanol (v/v) respectively in the same manner as in Example 3. After that, an S. aureus N315 strain was added to the BHI medium to which 1% glucose was added, and each of the dehydroabietic acid-supported nonwoven fabrics was immersed in the BHI medium, pressed with a stainless-steel circular washer, and allowed to stand at 37° C. for 24 hours. Thereafter, the biofilm formation inhibitory activity of the nonwoven fabrics was measured in the same manner as the allophane membrane of Example 3. The result is shown in FIG. 8.

### Example 4

The dehydroabietic acid-supported allophane membranes prepared by the AD method described above were washed with acetone. The number of washes was 0, 2, 4, and 6, respectively. The washing was carried out by immersing each dehydroabietic acid-supported allophane membranes in 0.75 mL acetone together with the base material and allowing it to stand for 30 minutes. Washing was carried out by pulling up each of the dehydroabietic acid-supported allophane membranes after each washing and immersing it in fresh acetone in the same manner.

Next, an S. aureus N315 strain was added to the BHI medium to which 1% glucose was added, the dehydroabietic acid-supported allophane membranes washed 0 times, 2 times, 4 times, and 6 times were immersed in the BHI medium together with the base materials, and each of the base materials was pressed with a stainless-steel circular washer and allowed to stand at 37° C. for 24 hours.

The biofilm formation amount of each of the allophane membranes was measured. Specifically, the amounts of biofilm formed on the surfaces of the allophane membranes differing in the number of washes were measured by the CV method, and the biofilm formation inhibitory activity of each allophane membrane was calculated. The result is shown in FIG. 9.

### Comparative Example 4

A dehydroabietic acid solution (solvent: acetone) was uniformly added dropwise to the nonwoven fabric (1 cm×1 cm) and dried overnight (final concentration: 25 μg/cm²). A plurality of dehydroabietic acid-supported nonwoven fabrics was prepared, and washed 0 times, 2 times, 4 times, and 6 times with acetone respectively in the same manner as in Example 4. After that, an S. aureus N315 strain was added to the BHI medium to which 1% glucose was added, each of the dehydroabietic acid-supported nonwoven fabrics was immersed in the BHI medium, pressed with a stainless-steel circular washer, and allowed to stand at 37° C. for 24 hours. Thereafter, the biofilm formation inhibitory activity of the nonwoven fabrics was measured in the same manner as the allophane membrane of Example 4. The result is shown in FIG. 9.

FIG. 7 is a scatter plot showing the biofilm formation inhibitory activity of the dehydroabietic acid-supported allophane membranes (Example 2) and the biofilm formation inhibitory activity of the dehydroabietic acid-supported nonwoven fabrics (Comparative Example 2) without forming an allophane membrane, and approximate curves of their exponential functions. In FIG. 7, the horizontal axis represents the number of washes, and the vertical axis represents the biofilm formation inhibitory activity. In FIG. 7, the biofilm formation inhibitory activity is expressed as a percentage (%) in the case where the biofilm formation inhibitory activity of a sample with 0 washes (in Example 2, the dehydroabietic acid-supported allophane membrane with 0 washes, and in Comparative Example 2, the dehydroabietic acid-supported nonwoven fabric with 0 washes) is set to 100. Referring to FIG. 7, the slope of the approximate curve was −0.006 for the allophane membrane and −0.011 for the nonwoven fabric. Although both the allophane membranes and the nonwoven fabrics had decreased biofilm formation inhibitory activity after washing with purified water, the biofilm formation inhibitory activity of the allophane membranes was kept higher than that of the nonwoven fabrics, and the biofilm formation inhibitory effect was maintained for a long time. As a result, it can be seen that the elution amount of dehydroabietic acid supported on the allophane membrane into water is smaller than the elution amount of dehydroabietic acid supported on the nonwoven fabric into water.

FIG. 8 is a scatter plot showing the biofilm formation inhibitory activity of the dehydroabietic acid-supported allophane membranes (Example 3) and the biofilm formation inhibitory activity of the dehydroabietic acid-supported nonwoven fabrics (Comparative Example 3) without forming an allophane membrane, and approximate curves of their exponential functions. In FIG. 8, the horizontal axis represents the number of washes, and the vertical axis represents the biofilm formation inhibitory activity. In FIG. 8, the biofilm formation inhibitory activity is expressed as a percentage (%) in the case where the biofilm formation inhibitory activity of a sample with 0 washes (in Example 3, the dehydroabietic acid-supported allophane membrane with 0 washes, and in Comparative Example 3, the dehydroabietic acid-supported nonwoven fabric with 0 washes) is set to 100. Referring to FIG. 8, the slope of the approximate curve was −0.04 for the allophane membrane and −0.048 for the nonwoven fabric. Both the allophane membranes and the nonwoven fabrics had decreased biofilm formation inhibitory activity after each washing with 70% ethanol. Although the biofilm formation inhibitory activity of the allophane membranes was kept slightly higher than the biofilm formation inhibitory activity of the nonwoven fabrics, there was no significant difference in the effect. As a result, it can be seen that the elution amount of dehydroabietic acid supported on the allophane membrane into ethanol and the elution amount of dehydroabietic acid supported on the nonwoven fabric into ethanol are substantially the same.

FIG. 9 is a scatter plot showing the biofilm formation inhibitory activity of the dehydroabietic acid-supported allophane membranes (Example 4) and the biofilm formation inhibitory activity of the dehydroabietic acid-supported nonwoven fabrics (Comparative Example 4) without forming an allophane membrane, and approximate curves of their exponential functions. In FIG. 9, the horizontal axis represents the number of washes, and the vertical axis represents the biofilm formation inhibitory activity. In FIG. 9, the biofilm formation inhibitory activity is expressed as a percentage (%) in the case where the biofilm formation inhibitory activity of a sample with 0 washes (in Example 4, the dehydroabietic acid-supported allophane membrane with 0 washes, and in Comparative Example 4, the dehydroabietic acid-supported nonwoven fabric with 0 washes) is set to 100. Referring to FIG. 9, the slope of the approximate curve was −0.1 for the allophane membrane and −0.312 for the nonwoven fabric. Although both the allophane membranes and the nonwoven fabrics had decreased biofilm formation inhibitory activity after each washing with acetone, the biofilm formation inhibitory activity of the allophane membranes was kept higher than that of the nonwoven fabrics, and the biofilm formation inhibitory effect was maintained for a long time. As a result, it can be seen that the elution amount of dehydroabietic acid supported on the allophane membrane into acetone is smaller than the elution amount of dehydroabietic acid supported on the nonwoven fabric into acetone.

Referring to the scatter plots showing the biofilm formation inhibitory activity of the dehydroabietic acid-supported allophane membranes and nonwovens shown in FIG. 7 to FIG. 9 and the slopes of the approximate curves of their exponential functions, the elution amounts of dehydroabietic acid supported on the allophane membranes or nonwovens into each solvent were acetone>ethanol (70%)>water. In addition, it was also found that the elution amounts of dehydroabietic acid supported on the allophane membranes into water (hydrophilic solvent) and acetone (lipophilic solvent) were significantly less than the elution amounts of dehydroabietic acid supported on the nonwoven fabrics into water and acetone. Therefore, it has been shown that the allophane membrane has a high capacity to hold dehydroabietic acid, especially for water (hydrophilic solvent) and acetone (lipophilic solvent), and can maintain the biofilm formation inhibitory activity for a longer time.

### <Antimicrobial Activity Test of Dehydroabietic Acid-Containing Zeolite Membrane>

In Example 1 to Example 4 described above, the biofilm formation inhibitory activity of the allophane membrane containing dehydroabietic acid-supported allophane particles as a biofilm formation inhibitor was described. However, the porous material capable of supporting the biofilm formation inhibitor is not limited to allophane. In the following, an antimicrobial activity persistence test of a biofilm formation inhibitor in the case where zeolite is used as a support material will be described.

### Example 5

A dehydroabietic acid-supported zeolite membrane was prepared. The zeolite membrane was formed by applying a paste containing Zeolite 4A, a solvent, and a varnish (manufactured by Shinagawa General Co., Ltd.) on a substrate by screen-printing (thickness: 10 μm) and baking (baking temperature: 350° C. to 600° C.).

A dehydroabietic acid solution (solvent: acetone) was uniformly added dropwise to the formed zeolite membranes (1 cm×1 cm respectively) and dried overnight (final concentration: 250 μg/cm²), to prepare three dehydroabietic acid-supported zeolite membranes. An S. aureus N315 strain was added to the BHI medium to which 1% glucose was added, and the prepared dehydroabietic acid-supported zeolite membranes were immersed in the medium together with the base materials, and each of the base material was pressed with a stainless-steel circular washer and allowed to stand at 37° C. for 24 hours.

After that, the biofilm formation amounts of the zeolite membranes were measured. Specifically, the amount of biofilm formed on the surface of the zeolite membrane was measured by the CV method, and the biofilm formation amount of the zeolite membrane was calculated.

### Example 6

A dehydroabietic acid solution (solvent: acetone) was uniformly added dropwise to the zeolite membranes formed in the same manner as in Example 5, and dried overnight (final concentration: 25 μg/cm²) to prepare three dehydroabietic acid-supported zeolite membranes. An S. aureus N315 strain was added to the BHI medium to which 1% glucose was added, and the prepared dehydroabietic acid-supported zeolite membranes were immersed in the medium together with the base materials, and each of the base material was pressed with a stainless-steel circular washer and allowed to stand at 37° C. for 24 hours. Thereafter, the biofilm formation amounts of the zeolite membranes were measured in the same manner as in Example 5.

### Example 7

A dehydroabietic acid solution (solvent: acetone) was uniformly added dropwise to the zeolite membranes formed in the same manner as in Example 5, and dried overnight (final concentration: 2.5 μg/cm²) to prepare three dehydroabietic acid-supported zeolite membranes. An S. aureus N315 strain was added to the BHI medium to which 1% glucose was added, and the prepared dehydroabietic acid-supported zeolite membranes were immersed in the medium together with the base materials, and each of the base material was pressed with a stainless-steel circular washer and allowed to stand at 37° C. for 24 hours. Thereafter, the biofilm formation amounts of the zeolite membranes were measured in the same manner as in Example 5.

### Reference Example 1

Three zeolite membranes formed in the same manner as in Example 5 were prepared as Reference Example 1. An S. aureus N315 strain was added to the BHI medium to which 1% glucose was added, the zeolite membranes were immersed in the medium together with the base materials, each of the base material was pressed with a stainless-steel circular washer, and allowed to stand at 37° C. for 24 hours. Thereafter, the biofilm formation amounts of the zeolite membranes were measured in the same manner as in Example 5.

The results of Examples 5 to 7 and Reference Example 1 are shown in Table 1 below. In Table 1, the biofilm formation amounts in Examples 5 to 7 are expressed as percentages (%) in the case where the biofilm formation amounts of Reference Example 1 are set to 100. Also, the biofilm formation amounts in Examples 5 to 7 and Reference Example 1 shown in Table 1 are the average of the biofilm formation amounts of the three zeolite membranes used in each of Examples and Reference Example 1.

TABLE 1

| | Concentrations of | Amounts of |
|---|---|---|
| | dehydroabietic acid | biofilm formation |
| | [μg/cm²] | [%] |
| Example 5 | 250 | 20.9 |
| Example 6 | 25 | 48.6 |
| Example 7 | 2.5 | 70.1 |
| Reference | 0 | 100 |
| Example 1 | | |

As is clear from Table 1, in Examples 5 to 7, it was shown that the higher the concentration of the supported dehydroabietic acid, the lower the biofilm formation amount of the zeolite membrane. Therefore, it was shown that zeolite has the ability to hold dehydroabietic acid, that is, a biofilm formation inhibitor, like allophane. Therefore, it is understood that the zeolite membrane containing zeolite exhibits the same biofilm formation inhibitory effect as the biofilm formation inhibitor-supported allophane membrane, depending on the concentration of the supported biofilm formation inhibitor.

In addition, when each BHI medium was observed after adding an S. aureus N315 strain to the BHI medium to which 1% glucose, immersing the zeolite membranes of Examples 5 to 7 and Reference Example 1 in the medium together with the base materials and allowed to stand at 37° C. for 24 hours, it was observed that the BHI medium of Reference Example 1 was entirely cloudy and a biofilm was entirely formed. On the other hand, it was shown that the BHI mediums of Examples 5 to 7 were less cloudly than the BHI medium of Reference Example 1, and in particular, the BHI medium of Example 5, in which the zeolite membranes with the highest concentration of supported dehydroabietic acid were immersed, had high transparency and it was shown that the zeolite membranes had a high bactericidal effect.

While embodiments of the present invention have been described above, it should be understood that modifications, variations, and changes can be made without departing from claims.

What is claimed is:

1. A coating comprising:
An antimicrobial porous membrane, the porous membrane including an allophane particle and an abietane-type diterpenoid compound adsorbed on the allophane particle.

2. The coating according to claim 1, wherein the abietane-type diterpenoid compound is at least one selected from among abietic acid, dehydroabietic acid and neoabietic acid.

3. The coating according to claim 1, wherein the antimicrobial porous membrane further includes an inorganic salt and/or at least one metal ion adsorbed on the porous particle.

4. The coating according to claim 3, wherein the inorganic salt and/or the at least one metal ion has an antimicrobial property.

5. The coating according to claim 3, wherein the at least one metal ion includes at least one of a platinum ion, a silver ion and a copper ion.

6. The coating according to claim 1, wherein the abietane-type diterpenoid compound is adsorbed directly onto the allophane particle without a holding material.

7. A fabric comprising:
a base material, the base material being a fabric; and
an antimicrobial porous membrane arranged on the base material, the antimicrobial porous membrane including an allophane particle and an abietane-type diterpenoid compound adsorbed on the allophane particle.

8. The fabric according to claim 7, wherein the base material is a nonwoven fabric.
